(19) United States
(12) Patent Application Publication
(10) Pub. No.: US 2013/0096486 A1
(43) Pub. Date: Apr. 18, 2013

(54) Activatable Drug Dispensing System

(71) Applicants: Britta Schroeder, Unterhaching (DE); Thomas Roos, Schillerstrasse (DE); Thomas Beckert, Birkenhard (DE)

(72) Inventors: Britta Schroeder, Unterhaching (DE); Thomas Roos, Schillerstrasse (DE); Thomas Beckert, Birkenhard (DE)

(73) Assignee: ACINO AG, Miesbach (DE)

(21) Appl. No.: 13/582,150

(22) PCT Filed: Mar. 2, 2011
(86) PCT No.: PCT/EP11/53136

§ 371 Date: Nov. 13, 2012

(30) Foreign Application Priority Data
Mar. 4, 2010 (DE) 1020100025984

(51) Int. Cl. A61N 1/30

(52) U.S. Cl. CPC A61N 1/303
USPC 604/20; 604/503

(57) ABSTRACT

The invention relates to a system (1) for controlling an electrolytic reaction in a reaction medium, with the system (1) comprising a primary energy storage (10), an intermediate storage (30) and a control unit (50). The control unit of the system is adapted for transferring electrical energy from the primary energy storage into the intermediate storage, monitoring the state of charge of the intermediate storage, establishing an electric connection between the reaction medium and the intermediate storage when the state of charge of the intermediate storage is larger than or equal to a first threshold value, and interrupting an electric connection between the reaction medium and the intermediate storage when the state of charge of the intermediate storage is smaller than or equal to a second threshold value. The invention further relates to an electrophoretic transdermal application system (40) comprising such a system (1) for controlling an electrolytic reaction for changing the pH value in the reaction medium of the matrix (43) of the electrophoretic transdermal application system (40), as well as to a method for transferring electric charges.

The invention relates to a system for controlling an electrolytic reaction in a reaction medium. The invention particularly relates to an electronically controlled energy supply system for the autonomous operation of an electrophoretic transdermal application system (TDS standing for “Transdermal Delivery System”).

Transdermal application systems serve for administering pharmaceuticals through the skin of a patient into his/her blood circuit. The transdermal administering of pharmaceuticals has a number of advantages as compared to conventional forms of administration. For example, active agents can be administered which are not taken up through the gastro-intestinal tract. The effectiveness of the administration is not affected by food intake and is not subjected to the first-pass effect of the liver. Further, it is not invasive and makes it possible to maintain a need-based level of active agent in the blood also over quite long periods of time.

The most important limiting factor for the administration of an active agent from a transdermal therapeutical system is the stratum corneum of the skin which the active agents must penetrate before they can percolate into the blood vessels which pervade the skin. In order to be able to adjust a desired level of active agents in the blood, a sufficiently high transport of the active agents through the skin must be ensured. The permeation of the active agent through the skin can be active or passive. In the case of an active transport of active agents a skin-permeating electric field is applied to the skin, along which the provided active agents are driven into the skin in ionic form. Since owing to the field also other ions present in the skin are transported, this mode of administration being called iontophoresis is often connected with skin irritations.

The passive transport of active agents is effected by the diffusion of the active agents along the active-agent concentration gradient which exists between the TDS and area of the skin pervaded by blood vessels. The flow of active agent is determined by the concentration of the active agent at the skin surface. Since the diffusion-determined delivery of active agent from a TDS leads to a reduction of the concentration of active agent in its regions facing the skin, the active agent concentration available at the skin surface and, in connection therewith, also the delivery rate of the active agent to the blood circuit of a patient decreases over time. For a long-term application of a TDS the concentration of active agent in the matrix must therefore be chosen appropriately high. Accordingly high is the residual amount of active agent which remains in the active-agent reservoir of a TDS after coming below the minimum concentration of active agent at the skin side, which is necessary for the application.

In order to be able to maintain the concentration of active agent at the skin surface at a sufficiently high level also over quite long periods of time of administration, electrophoretic transdermal application systems are used in which the active agents within the reservoir are driven to its side facing the skin by way of an electric field. For this purpose, the active agents have, of course, to be present in ionic form. In these electrophoretic transdermal systems the electric field is generated by two electrodes which, as in the case of a capacitor, are arranged opposite to each other. The reservoir of the active agent is located between the two electrodes. In order to enable the emission of active agent, the electrode at the side of the skin is adapted to be permeable for the active agent.

The described electrophoretic transdermal systems make it possible to maintain a sufficiently high concentration of active agent at the skin side also over quite long periods of application, but the administration of active agents through these systems cannot be controlled to the therapeutically desired extent.

It is therefore desirable to provide an electrophoretic transdermal application system which makes possible a controlled release of active agents at its side facing the skin.

Embodiments of electrophoretic transdermal application systems for the specific control of the delivery of active agent have a matrix comprising the active agents in ionic form, which is located between two electrodes. The matrix composition and the electrode setup are selected in such a way that, when an electric voltage is applied to the electrodes, electrochemical reactions are triggered which change the pH value in the reaction medium of the matrix and thereby convert some of the active agents contained in the matrix from their ionic form into their neutral form. The conversion is effected in such a way that the concentration of the neutral active agents at the skin-facing side of the system is sufficiently high for obtaining the desired level of active agents in the blood, and at the same time in such a way that the concentration of the neutral active agents exceeds at no place within the matrix a value starting from which their solubility in the matrix is exceeded. The concentration of the neutral active agents is a function of the pH value prevailing in the matrix, more exactly of the pH value in the regions of the matrix in which the neutral active agent is present. If the concentration of the neutral active agent exceeds the valid maximum value, i.e. the saturation concentration, the active agent becomes immobile because of the precipitation and can no longer diffuse towards a patient's skin.

It is therefore desirable to provide a device for controlling the electrochemical conversion in such an electrophoretic transdermal application system.

Since, generally, electrophoretic transdermal application systems are applied to the patient's skin over periods of several hours to several days, it is further desirable, in order not to restrict the freedom of movement of the patent, to provide an autonomous energy supply for such a system, which allows the patient to move during the entire time of application independent of a network-connected energy supply.

Therefore, embodiments of the invention relate to a system for controlling an electrolytic reaction in a reaction medium, with the system comprising a primary energy storage, an intermediate storage and a control unit. The control unit of the system is designed for the purpose of transferring electric energy from the primary energy storage into the intermediate storage, of checking the state of charge of the intermediate storage and of establishing or interrupting the electrical connection between the reaction medium and the intermediate storage in dependence on the state of charge of the intermediate storage. An electrical connection between the reaction medium and the intermediate storage is established when the state of charge of the intermediate storage is larger than or equal to a first threshold value, and is interrupted when the state of charge of the intermediate storage is smaller than or equal to a second threshold value.

Such a system makes possible an exact control of the charge transferred to the reaction medium and thereby a precise control of the electrochemically converted amount of substances in the reaction medium. The system also makes possible a control of the temporal course of the conversion of the substances. The decoupling of the primary energy storage and the supply of charge to the reaction medium allows selecting energy storages which exhibit a high capacity while having a small construction volume, but otherwise are not suitable to the requested extent for maintaining the electrolytic reaction. Among these, there is particularly the suitability for introducing the amount of charge necessary for the controlled course of the electrolytic reaction into the reaction medium while maintaining the minimum electrode voltage necessary for the progress of the reactions.

Further embodiments of the invention also relate to an electrophoretic transdermal application system which comprises a first electrode, a second electrode and a matrix located between the first electrode and the second electrode as well as a system as indicated above for controlling an electrolytic reaction in a reaction medium. The matrix which is in contact with the electrodes comprises at least one active agent, the ratio of which of its neutral form to its ionic form changes with the pH value of the matrix. The second electrode of the electrophoretic transdermal application system is arranged at the skin-side surface of the matrix, i.e. at the surface of the matrix which faces the patient's skin when the system is applied, and is furthermore adapted to be permeable for the at least one active agent in its neutral form, which is contained in the matrix. Furthermore, the system for controlling an electrolytic reaction in the reaction medium is electrically connected with the first electrode and the second electrode in such a way that at least the electric connection to one of the electrodes is closed and interrupted by the control unit of the system in dependence on the state of charge of the intermediate storage.

In preferred embodiments, the primary energy storage is formed by a galvanic cell or an array of galvanic cells. The use of galvanic cells makes possible a network-independent energy supply of the reaction medium of the matrix, so that it is ensured that the patients can move freely. Advantageously, in embodiments thereof lithium-manganese dioxide cells are used as primary energy sources, since the latter exhibit a high energy density, i.e. a high battery capacity, while having a small construction volume, and with a rated voltage of 3 V they provide a sufficiently high voltage for causing an electrolytic reaction in the reaction medium of the matrix. It should be noted that in this publication the expression battery is used in a colloquial and not in a technical sense, i.e. it describes one single galvanic cell as well as an array of galvanic cells connected to each other.

Further embodiments advantageously comprise an energy transfer device being adapted for transferring electric energy from the primary energy storage to the intermediate storage, the energy being transferred by means of a charge current which does not exceed a pregiven value. In this way it is guaranteed that an energy withdrawal from the primary energy storage does not reach or exceed values which could affect its life time or even lead to its premature destruction. In this way the threats to a user, e.g. due to an overheating of the primary energy storage or an uncontrolled progress of the electrolytic reaction is reliably forestalled. The transfer of electric energy from the primary energy storage to the intermediate storage is preferably effected by a constant charge current, so that the withdrawal of energy from the primary energy storage can largely be kept independent of its respectively current discharge state.

In a further advantageous embodiment, the control of the system is further adapted for determining the state of charge of the intermediate storage by way of a voltage applied to the intermediate storage, so that the amount of charge transmitted to the reaction medium can be controlled in an easy way.

According to a further embodiment, the first and the second threshold value for the state of charge of the intermediate storage represent values that are dependent on the operating voltage of the primary energy storage. This makes possible an advantageous adaptation of the energy amount respectively transferred from the primary energy storage to the intermediate storage in one cycle and of the amount of charge transferred from the intermediate storage to the reaction medium to the respectively current discharge state of the primary energy storage.

For preventing the voltage from falling below the minimum voltage necessary for maintaining the electrolytic reaction, in a preferred embodiment thereof the control unit is adapted for interrupting an electric connection between the reaction medium and the intermediate storage, when the second threshold value falls below a predetermined threshold value. In an advantageous embodiment thereof the control unit is further adapted to determine a falling below the predetermined threshold value through the operating voltage of the primary energy storage, so that the decision whether the transfer of charge to the reaction medium is continued is made on the basis of the current operating state or the discharge state of the primary energy storage.

According to further advantageous embodiments, the system further comprises a reference voltage generator for generating a reference voltage which shows a reference voltage of a constant value, which is independent of changes in the operating voltage of the primary energy storage. Such a reference voltage provides a standard voltage which makes possible a precise adjustment and measurement of electric quantities appearing in the system. In a preferred embodiment, the energy transfer device is adapted for using the reference voltage for controlling the charge current. In a further advantageous embodiment the control is adapted for determining the respectively current operating voltage of the primary energy storage by using the reference voltage.

In a preferred embodiment, the intermediate storage is formed by a capacitor.

The withdrawal of charge from a capacitor can be effected by substantially higher currents than from a primary energy storage, the withdrawn amount of charge and the voltage with which the charge is drawn being monitorable and controllable in an easy way. Since the voltage applied to a capacitor is proportional to the charge stored therein, the amount of charge withdrawn from the capacitor can be determined in a simple way through the capacitor voltage. Moreover, in this way it can be ensured that the electrode voltage, when the charge is transferred into the reaction medium, does not fall below the minimum voltage necessary for the electrolytic reaction.

In a further preferred embodiment the control is adapted for determining an amount of charge transferred into the reaction medium while an electrical connection between the intermediate storage and the reaction medium has been established. This can, e.g., be effected, as it is described in more detail further below, by the integration of the charge current by which the energy is transferred from the primary energy storage into the intermediate storage, or by the first and the second threshold values through which the connection of the intermediate storage to the reaction medium is controlled.

For controlling as well as recording the amount of charge introduced into the reaction medium, the system further comprises, according to a further preferred embodiment, a storage device adapted for storing data which relate to the amount of charge introduced into the reaction medium during the existence of one or more electrical connections to the intermediate storage. These data can relate, on the one hand, to the amounts of charge introduced into the reaction medium during the individual charge transfer cycles, as well as to the amount of charge accumulated during a plurality of cycles. Moreover, these data can also relate to periods of time in which a charge is introduced or is not introduced, and, of course, also to different amounts of charges at different times.

For a simple handling, the system further also comprises, according to a further embodiment, a user interface adapted for activating the system through a user. For informing the user on the state or the progress of the application, the user interface—in a further embodiment thereof—is further adapted to indicate information to the user, e.g. by activating an illuminant, by activating a data display device or by activating an electro-acoustic or electro-mechanic device.

Further features of the invention are inferable from the following description of exemplary embodiments in connection with the claims as well as the Figures. The features indicated for the described exemplary embodiments can be implemented in an embodiment according to the invention in other combinations and, in particular, also for themselves. In the following explanation of some exemplary embodiments of the invention reference is made to the enclosed Figures, in which

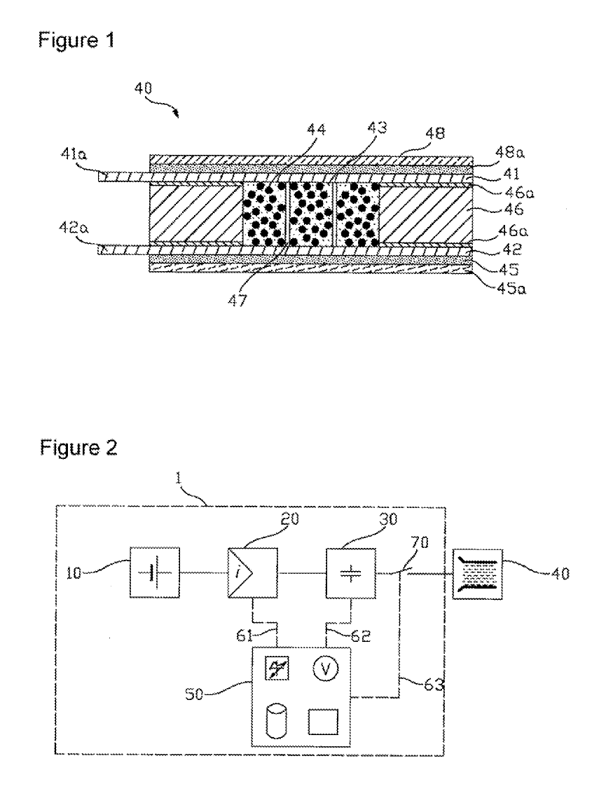

FIG. 1 shows a cross-section through an electrophoretic transdermal application system for the use in an arrangement according to FIG. 2 in a schematic representation,

FIG. 2 shows a schematic circuit diagram of a system for controlling an electrolytic reaction for changing the pH value in a reaction medium,

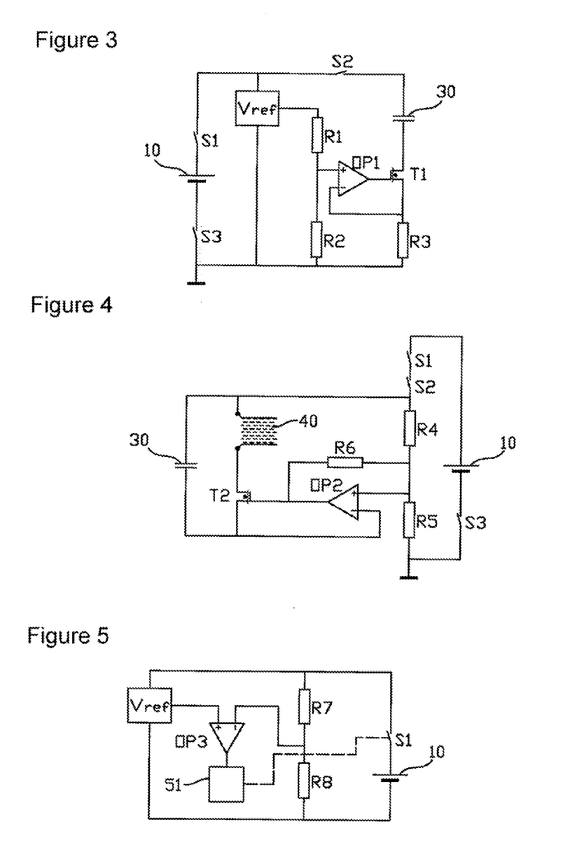

FIG. 3 shows a circuit diagram for illustrating an embodiment of an energy transfer device, and

FIG. 4 shows a circuit diagram for illustrating a voltage-controlled switching element for the transfer of charge from an intermediate storage into an electrophoretic transdermal application system,

FIG. 5 shows a circuit diagram for illustrating an example for a threshold value control circuit,

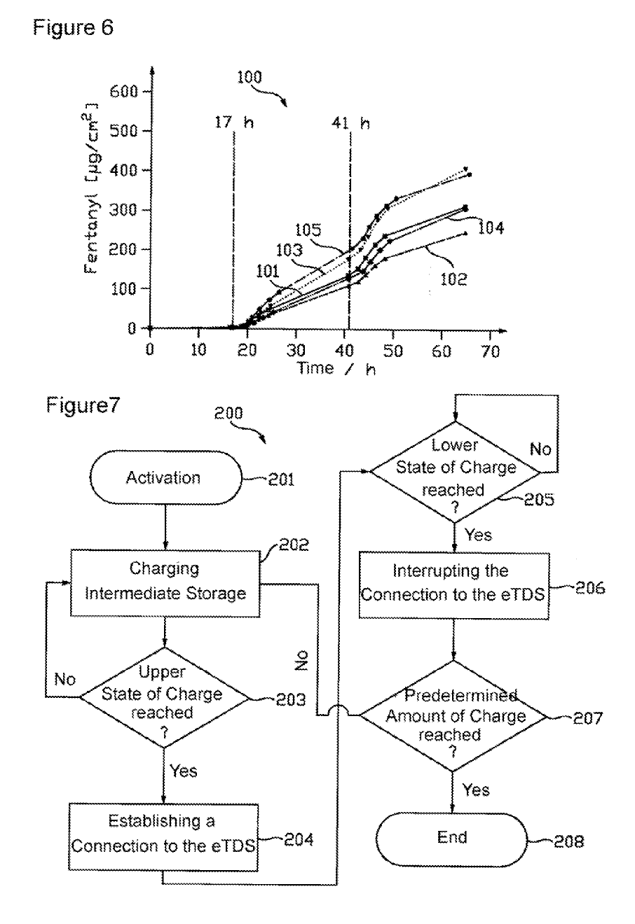

FIG. 6 shows a diagram with skin permeation profiles of tests which were made by using an electrophoretic transdermal application system according to FIG. 1 with a system according to FIG. 2, and

FIG. 7 shows basic steps of a method for transferring charge from a primary energy storage into an electrophoretic transdermal application system according to FIG. 1.

The electrophoretic transdermal application system 40 shown in cross-section in FIG. 1 has two electrodes 41 and 42 between which a matrix 43 is located. The matrix 43 is in contact with the two electrodes and comprises the active agent(s) 44. The lower surface of the electrophoretic transdermal application system 40 with regard to FIG. 1 is intended to contact the skin of a user. In the following it is called skin-side surface of the transdermal application system 40. At the skin-side surface of the electrode 42 an adhesive layer 45 is attached, which serves for fixing the electrophoretic transdermal application systems 40 to a skin surface.

The skin-side electrode 42 shows openings through which the active agent can enter the adhesive layer 45. As a protection against contamination, when it is not in use, the adhesive layer 45 can be covered with a removable release liner 45a. The counter-electrode 41 can be provided with openings—just like the skin-side electrode 42 facing the latter—but can also have a closed surface. The side of the counter-electrode 41 facing away from the skin is covered by a cover foil 48, which is attached to the electrode 41 by an adhesive layer 48a.

The side faces of the matrix 43 arranged between the electrodes are preferably enclosed by a spacer 46 which counteracts a leaking of active agent 44 or of the matrix 43 out of the system and ensures that the two electrodes 41 and 42 do not come into contact with each other. If spacers of non-adhesive materials are used, the surfaces of the spacers 46 which are in contact with the electrodes can be provided with an adhesive layer 46a. If the matrix 43 spans a larger area, the distance between the electrodes in the range of the matrix is suitably maintained with the help of one or more additional support elements 47.

The matrix can be present as a liquid, in gel form or as a self-supporting solid. In the case of a flowable matrix a leaking of the matrix can also be prevented by other measures than those mentioned above. For example, the matrix can be embedded into a sheath, the electrode-facing surfaces of which are designed as a membrane. A thickening of the matrix with the help of suitable gelling agents or also other measures known to the skilled person are possible as well.

The electrode 42 and the counter-electrode 41 comprise terminals 42a and 41a, respectively, for the connection to an electric supply system. The electric supply system provides charge carriers having an electric potential which causes an electrolytic reaction in the reaction medium located between the electrodes.

The described structure of the transdermal application system 40 serves for transporting one or more of the active agents 44 contained in the matrix 43 through the skin-side electrode 42 and the adjoining adhesive layer 45 in a sufficient concentration and in a controlled way over an extended period of time to the surface of a skin on which the electrophoretic transdermal application system 40 is attached by means of the adhesive layer 45.

Preferably the matrix is a polymer-based matrix; for manufacturing the same basically all polymers can be used which are deployed in the production of transdermal systems and which are physiologically harmless, provided that they are hydrophilic or possibly amphiphilic and either contain water or can absorb water. Examples for such suitable polymers are indicated in the international patent application WO09/000,262. The term “matrix” as it is used in this application is to be understood in such a way that either the matrix already contains water and is storable as such, or, by introducing water shortly before the application of the system 40, possibly by water absorption from the skin.

In the matrix 43 there are one or more active agents 44, these active agents can either be alkaline or acidic. Respective active agents are characterized in that they can be converted into a ionic form. The active agents can be contained in the matrix in the form of their pharmaceutically acceptable salts. Examples for suitable active agents can be inferred from the international patent application No. WO09/000,262.

When using an alkaline active agent the pH value in the matrix is set so as to be smaller than the pKa value of the active agent. Thus, the active agent is mainly present in protonated form as cations. When an acidic active agent is used, the pH value in the matrix is correspondingly set so as to be larger than the pKa value of the active agent. In this way it is ensured that the active agent is largely present in deprotonated form as anions. The high percentage of active agent in ionic form guarantees that the active agent is present in dissolved form. Namely, the neutral form of the active agent is not readily soluble in water and can precipitate out of the solution if a particular concentration is exceeded. Thereby the active agent in the matrix would be immobilized and would no longer be freely available.

For the transport through the possibly provided adhesive layer 45 and the skin of a user resp. patient the active agent 44 should be present in neutral form. The conversion of a part of the ionic active agent 44 contained in the matrix is effected by applying a voltage to the electrodes 41 and 42. In this way not only an electric field permeating the matrix 43 is generated which leads to a migration of the ions contained therein, but also, above all, electrochemical processes at the electrode surfaces are triggered.

When alkaline active agents are used, the electrons injected into the system by the cathode react with the present oxonium ions (H₃O⁺) to form molecular hydrogen, and in the direct vicinity to the cathode there remain hydroxide ions (OH⁻). The hydroxide ions react with the protons at the protonated active agent, so that the active agent is deprotonated and is converted into its neutral form. Through the locally increased OH⁻-concentration an increase of the pH value takes place. In order to prevent the deprotonated active agent from crystallising out and, thus, from not migrating to the skin surface, the pH value should at no place within the matrix 43 rise above the pKa value of the active agent 44.

At the anode the hydroxide ions emit electrons, so that molecular oxygen is generated, and at the electrode there remain oxonium ions. Gas bubbles which are generated in this process can affect a controlled application of the transdermal application system 40. In order to prevent this, the upper electrode 41 acting as the anode and the cover foil 48 located thereabove can be designed so as to be permeable to gas. Suitable cover foil materials can be inferred from the international patent application WO09/000,262.

The formation of gas bubbles can be largely reduced by using a silver anode in connection with a sufficient amount of chloride in the matrix. Namely, in this case, at the anode hardly any oxygen is generated, but in the first place silver is oxidized, according to the reaction Ag+Cl⁻→AgCl+e⁻ thereby forming silver chloride which is deposited at the anode surface. In this context the term silver anode is understood to be an anode which either consists of silver or has at least at the surface facing the matrix a silver coating. As silver anodes in particular silver-coated stainless steel fabrics, but also perforated silver foils or silver-coated polyester fabrics are preferred.

Due to the reduced generation of oxonium ions in the anode region when silver anodes are used, the pH value also rises in the proximity of the anode, so that the deprotonation of alkaline active agents extends over the entire matrix region. In this special case, in contrast to the representation in FIG. 1, it is therefore also possible to operate the anode as the skin-side electrode. This would have the additional advantage that hydrogen generated at the cathode can degas through the cathode and the cover foil without having to migrate through the matrix.

Preferably, cathode and anode are designed as gas-permeable silver electrodes, i.e. are made of silver or a silver-coated material. In principle, however, as electrodes all conventional noble metal electrodes come into question, such as electrodes consisting of or being coated with gold, platinum or palladium, as well as stainless steel or copper electrodes or copper-coated electrodes. Further suitable electrodes are those based on carbon. The anode and the cathode can, of course, also be made of different materials.

The electrodes, again, can be designed as fabrics, preferably as grid-shaped fabrics, or as perforated or porous foils or as foils with a conductive material printed or printed in a pattern thereon. In particular, the upper electrode 41 can be designed as a conductive patterned imprint on the cover foil. When a grid-shaped electrode or an electrode with a grid printed thereon is used, the mesh size can, e.g. be 0.001 to 1 mm and, in particular, 0.01 to 0.05 mm.

FIG. 2 shows a schematic circuit diagram of an electric supply system 1 for an electrophoretic transdermal application system 40 as described above. The electric supply system 1 is designed as a system 1 for the control of an electrolytic reaction in a reaction medium. The reaction medium is part of the matrix 43. The electric supply system 1 comprises a primary energy storage 10, an intermediate storage 30 and a control unit 50. In the shown embodiment an energy transfer device 20 is provided for the energy transfer from the primary energy storage 10 to the intermediate storage 30.

The purpose of the supply system 1 is the introduction of metered amounts of charge into the electrophoretic transdermal application system 40 while maintaining a minimum electrode voltage. Such an introduction of charge at a voltage above a minimum electrode voltage leads to an electrolytic reaction progressing in a controlled way in the reaction medium of the matrix 43 and, consequently, to a conversion of a certain amount of the active agent contained in the matrix from its ionic form to its neutral form.

In the shown exemplary embodiment electric energy is drawn from the primary storage 10 under defined conditions and is transferred into the intermediate storage 30. In the embodiments, the withdrawal of energy can be autonomous or—as in the shown example—can be effected in a controlled way via the control connection 61. The amount of energy transferred into the intermediate storage 30 is monitored by the control unit 50 through the measurement connection 62 on the basis of the state of charge of the intermediate storage 30. If the state of charge of the intermediate storage 30 has reached a predetermined first value, the control unit 50 generates an electric connection from the intermediate storage 30 to the electrophoretic TDS 40 by means of a switching element 70 controlled by the control connection 63, and in this way enables the transfer of charge into the reaction medium of the matrix 43. In order to ensure that the charge is always introduced into the reaction medium at an electrode voltage sufficiently high for the electrolytic conversion, the control unit 50 interrupts the electric connection as soon as the state of charge has fallen below a predetermined second value.

The primary energy storage 10 is preferably a battery, and for obtaining a compact structure of a supply system 1 batteries are preferred that only contain one galvanic cell. If the electrolytic reaction, however, requires higher voltages, also batteries that comprise a plurality of galvanic cells or several batteries connected in series can be used. In an electrophoretic TDS 40 as described above the minimum electrode voltage for maintaining the electrolytic reaction is usually about 2 V, so that as a primary energy storage preferably batteries with small dimensions and voltages of considerably more than 2 V are used. Particularly preferable are lithium manganese dioxide cells and, among these, particularly so-called lithium button cells. Such cells have a high gravimetric energy density of presently about 250 to 300 Wh/kg and have a rated voltage of 3V. The self-discharging of lithium button cells being less than 1% per year is very low, so that an electric supply system 1 equipped therewith can be stored for a long time; at room temperature up to ten years. Furthermore, the battery voltage is constant up to the almost complete discharge of the battery provided that the maximum discharge current is not exceeded, and, thus, allows to almost completely use the battery capacity. The compact structure of lithium button cells makes further possible to integrate the electric supply system 1 and the electrophoretic TDS 40 within a single patch assembly.

The current with which energy can be drawn from galvanic cells is, however, limited. At too high discharge currents not only the battery voltage and the battery capacity of the cell quickly decrease, but the cell also heats up, what can, among others, result in leakages or a bursting of the battery and thereby in burns and chemical burns of the skin. Due to the low internal resistance of an electrophoretic transdermal application system 40, the battery 10 cannot directly be connected to the TDS 40. Though, in this case, the discharge current could be lowered to values compatible with the battery by means of an impedance connected in series to the electrophoretic TDS 40, the electrode voltage at the TDS 40 would decrease to values below the minimum electrode voltage necessary for the electrolytic reaction because of the voltage drop at the impedance. In the following this minimum electrode voltage is called threshold voltage.

For making possible the charge input into the electrophoretic transdermal application system 40 at voltages above the minimum electrode voltage without excessively overloading the battery 10, an intermediate storage 30 is provided in the electric supply system 1, from which charge with higher current intensities can be drawn than can from the battery. Preferably a capacitor is used for serving as the intermediate storage, since the latter stores energy at negligible losses and is not affected by the currents occurring at the charge transfer.

The transfer of the electric energy from the primary energy storage 10 into the intermediate storage 30 is effected by means of the energy transfer device 20. The energy transfer device 20 is designed in such a way that charge is drawn from the primary energy storage 10 always with current intensities that do not exceed the maximally admissible discharge current intensity. In the simplest case an electric or ohmic resistor serves as the energy transfer device 20. The capacitance of the capacitor and the value of the resistor are matched in such a way to the voltage of the primary energy storage 10 that the capacitor can be charged within a certain period of time to a voltage above the threshold voltage and having a sufficient distance thereto.

In a preferred embodiment of the electric supply system 1, the energy transfer device 20 comprises a constant current control which guarantees a charge transfer from the primary energy storage 10 into the intermediate storage 30 with a constant current. FIG. 3 illustrates an example of a circuit for the transfer of the charge from the primary energy storage 10 into the intermediate storage 30 with a constant current. The switching elements S1, S2 and S3 represent controllable switching elements through which the connection to the battery 10 can be interrupted at various locations of the circuit by the control unit (not shown in the Figure). The switching elements are preferably implemented in the form of electronic components, e.g. of MOSFETs, and serve for controlling the energy transfer in dependence on actions of the user, conditions of the control as well as of the current battery voltage. However, the switching elements have no influence on the function of the constant current drain.

The battery voltage of the primary energy storage 10 is converted into a reference voltage by the reference voltage generator Vref, which has a constant value independent of variations of the battery voltage. The reference voltage is applied to the voltage divider formed by the resistors R1 and R2. In the main circuit the capacitor serving as the intermediate storage 30 is connected to the battery 10 in series with a transistor T1, e.g. an n-channel MOSFET, and a measuring resistance R3. The voltage drop at the measuring resistance R3 caused by the capacitor's charge current is compared in the operational amplifier OP1 to the stabilized voltage tapped at the voltage divider. The output signal generated by the operational amplifier OP1, which corresponds to a multiple of the difference of the compared voltages, is applied to the control electrode of the transistor T1 for controlling the current flow therethrough. If the voltage drop at the measuring resistance R3 is larger than the stabilized voltage tapped at the voltage divider, the current flow through the resistor T1 is reduced. In the reverse case, it is increased. In cooperation with the battery, the components R1, R2, R3, Vref, OP1 and T1 of the energy transfer device 20 cooperating as explained thus form a constant current source which, independently of the charge state of the capacitor 30, transfers equal amounts of charge in equal time units into the capacitor.

The measuring resistance R3 of the constant current source is designed in such a way that the capacitor can be charged to sufficiently high voltages for performing the electrolytic reactions in the electrophoretic transdermal application system 40. At a battery voltage of 3 V available from lithium cells, the resistance is, e.g., selected in such a way that the capacitor can be charged to 95% of the battery voltage, i.e. to a voltage of 2.85 V. Since the charge Q_{K} stored in a capacitor is directly related to the voltage U_{K} applied to the capacitor via the capacitance C_{K} thereof, according to the equation

Q_{K}=C_{K}·U_{K} (1),

the amount of charge to be stored in the capacitor can simply be adjusted through its capacitance and charging voltage.

If, e.g., the capacitor is cyclically charged to a voltage of 2.85 V, and subsequently discharged again to a voltage of 2.265 V, the charge output during one cycle is Q_{Z}=C_{K}·(0.585). At a capacitance of the capacitor of C=47 μF a cyclically output amount of charge of Q=27.5·10⁻⁶ C is obtained. Generally, the amount of charge ΔQ_{K} output by the capacitor per cycle is calculated from the voltage level difference ΔU_{K}, i.e. from the difference of the capacitor voltages at the beginning and at the end of a discharge cycle, according to:

ΔQ_{K}=C_{K}·ΔU_{K} (2).

Further, the transfer of charge from the primary energy storage 10 into the intermediate storage 30 can be effected in a time-controlled way. E.g., the control can be adapted for activating, after an activation of the electric supply system 1, possibly with a predetermined time lag, the charge transfer from the primary energy storage 10 into the intermediate storage 30 and further on to the electrophoretic TDS 40, and for continuing the transfer until a predetermined amount of charge has been introduced into the electrophoretic transdermal application system 40. Thereby a desired concentration of the active agent at the skin surface of a patient can be adjusted. Subsequently the charge transfer can be interrupted for a certain period of time and, after the expiration of this period, is resumed for a further time interval. This intermittent charge transfer can be repeated, if required, and the periods between the charge transfer intervals as well as the duration of these intervals can vary from repetition to repetition. In other embodiments, the control unit reduces, as soon as the amount of charge necessary for building up the initial concentration of active agent has been transferred, the amount of charge transferred on an average per unit of time to, e.g., values at which the concentration of active agents on the skin of the patient is kept substantially constant or is changed according to a predetermined profile. Such a control is preferably implemented in the form of a software-controlled device in the control unit, which is symbolized in FIG. 2 by a variable curve diagram.

The charge state of the capacitor serving as the intermediate storage 30 is preferably monitored through its voltage. For controlling the charging and discharging of the capacitor the control unit 50 comprises a voltage-controlled switching device which is symbolized in FIG. 2 by a V surrounded by a circle and is illustrated in FIG. 4 by a circuit diagram.

The voltage-controlled switching element is designed as a Schmitt trigger, i.e. in the form of an electronic circuit which has different switch-on and switch-off thresholds. Since the operational amplifier OP2 typically has a high amplification of about 5·10⁵, the voltage at the output of the operational amplifier varies in dependence on the voltage difference at its inputs between a low level 0V and a high level which approximately corresponds to its supply voltage, i.e. the battery voltage minus possible voltage drops at switching elements such as S3. Possible offset voltages can be compensated or minimized by suitably dimensioning resistors R4, R5 and R6.

At a low voltage level at the output of the operational amplifier OP2 the resistors R5 and R6 are effectively connected in parallel. The first threshold voltage supplied in this case to the positive input of the operational amplifier is therefore determined by a voltage divider which results from connecting the resistor R4 in series with resistors R5 and R6 connected in parallel with each other. The voltage supplied to the positive input of the operational amplifier is thereby lower—in respect of the ground potential of the circuit—than is the voltage tapped at the capacitor 30 and supplied to the negative input of the operational amplifier. The switching transistor T2 blocks, i.e. the switching element formed by the latter is open and prevents the charge from being discharged from the capacitor into the electrophoretic transdermal application system 40.

With increasing capacitor voltage the voltage supplied to the negative input of the operational amplifier OP2 decreases relative to the ground potential of the circuit, until it finally reaches and drops below the value of the first threshold voltage, so that the output potential of the operational amplifier OP2 switches to the high level. At this level, the resistors R4 and R6 are effectively connected in parallel. The second threshold voltage supplied in this case to the positive input of the operational amplifier is therefore determined by a voltage divider which results from a connection of resistor R5 in series with resistors R4 and R6 connected with each other in parallel, so that the potential applied to the positive input of the operational amplifier is increased relative to the ground potential and assumes a higher value than the voltage tapped at the capacitor 30 and supplied to the negative input of the operational amplifier. The switching transistor T2 is now conductive, i.e. the switching element formed by the latter is closed, and charge is discharged from the capacitor 30 into the electrophoretic transdermal application system 40.

Thus, with the described Schmitt trigger formed of the components OP2, R4, R5 and R6 an electric connection is formed between the capacitor serving as the intermediate storage 30 and the electrophoretic TDS 40, as soon as the state of charge of the capacitor reaches or exceeds the first threshold value determined by the first threshold voltage. As soon as the state of charge of the intermediate storage 30 has hereby dropped to a second threshold value determined by the second threshold voltage or has come below the same, the Schmitt trigger interrupts the electric connection, so that the intermediate storage can be recharged.

At a constant battery voltage of the primary energy storage 10 the first and the second threshold voltage are constant, so that at each connection of the capacitor with the electrophoretic TDS 40 equal amounts of charge are transferred into the reaction medium of the electrophoretic transdermal application system. The amount of charge introduced into the electrophoretic TDS 40 over a predetermined period of time can, therefore, easily be determined through the number of discharge cycles of the capacitor 30 and through the amount of charge transferred per discharge cycle according to equation 2.

For keeping constant the amount of charge introduced per discharge cycle into the electrophoretic transdermal application system 40 also at varying battery voltages, the voltage divider formed of the resistors R4, R5 and R6 can be supplied with a constant reference voltage which is generated by an above described reference voltage generator.

A further simple possibility of determining the amount of charge introduced into an electrophoretic TDS during a predetermined period of time is the use of a constant-current energy transfer device as described above. In this case the capacitor is always supplied with an amount of charge being constant per unit of time. Since this charge supply is not even interrupted during the charge transfer into the electrophoretic transdermal application system 40, the amount of charge altogether introduced into the system 40 can easily be calculated by multiplying the constant charging current by the time that has passed since the first exceeding of the lower second threshold value for the state of charge of the capacitor.

In the voltage-controlled switching device illustrated in FIG. 4, the electric connection from the intermediate storage 30 to the electrophoretic transdermal application system 40 is interrupted as soon as the voltage at the intermediate storage 30 falls below a threshold value, above called second threshold value. This threshold value is determined by the above described second threshold voltage, the absolute value of which changes with the battery voltage and becomes lower towards the end of the battery life. In order to prevent that hereby the lower threshold value for the state of charge of the intermediate storage 30 falls below a value at which the minimum electrode voltage is no longer reached and, consequently, an electrolytic reaction can no longer take place, the control unit is adapted for interrupting the electric connection as soon as the second threshold value dependent on the battery voltage falls below a threshold value determined by the minimum electrode voltage. Preferably this threshold value is determined based on the operating voltage of the primary energy storage 10, i.e. the respectively current battery voltage. An example for such a threshold value control is illustrated in FIG. 5. By means of a voltage divider formed of the resistors R7 and R8 a measuring voltage directly connected with the operating voltage is generated, which is compared, in a comparator circuit OP3, to the reference voltage of the reference voltage generator described above with reference to FIG. 3. Preferably the measuring voltage is adapted in such a way that, when the operating voltage falls below the admissible minimum value, the measuring voltage corresponds to the reference voltage. Of course, instead of the reference voltage also a voltage derived therefrom by a further voltage divider can be used. The output of the comparator OP3 can serve either directly for the control of the switching element S1, or, as shown in FIG. 5, can serve as the input value of a control 51 for driving the switching state of S1. The switching element S1 is preferably located in the main circuit of the electric supply system 1, so that the primary energy storage is electrically disconnected from the further components of the supply system at less than the minimum operating voltage.

Other embodiments of the control unit 50 comprise one or more analogue-to-digital-converters for converting analogue voltage values into digital values. In these embodiments, the operating or measuring voltage resp. the second threshold voltage is digitalized and compared to the values necessary for maintaining the minimum electrode voltage. For minimizing measurement inaccuracies caused by variations in the operating voltage, the digitalized voltage values can be normalized by the value respectively promptly obtained from the digitalization of the reference voltage.

The control unit 50 further comprises a software-controlled device for the sequence control, which is symbolized by a rectangle in FIG. 2. The software-controlled device accesses a storage device, which is symbolized by a drum in FIG. 2.

Furthermore, the electric supply system 1 comprises a user interface not shown in the Figures, which allows a user to activate the system 1. For the feedback to the user, the user interface can also comprise signalling devices through which the control unit makes information available to a user through optical, acoustic, and mechanical signals. As optical signalling devices illuminants, such as LEDs (light emitting diodes), or display units, e.g., small liquid crystal displays, are suitable. As examples for acoustic signalling devices electro-acoustic transducers, such as electrostatic sound transducers, can be mentioned, and for mechanical signalling devices electromechanical transducers which generate mechanical vibrations when electrically excited.

Preferably, the user interface comprises a push-button which, in the closed state, establishes an electric connection to the software-controlled device of the control unit 50. In this way the software-controlled device is activated.

The user interface further comprises a switching element S1 which is closed by the software-controlled control device, as soon as the latter is started, and hereby closes the main circuit independently of the switching state of the push-button. In a special embodiment of the control unit, the software-controlled device is divided into two sub-units, the first sub-unit closes the switching element S1 and the second sub-unit closes a switching element S2, which provides the voltage supply to the energy transfer device 20 and to the voltage-controlled switching element.

In the storage devices preferably those values are stored that specify the charge supply of an electrophoretic transdermal application systems 40 by means of the electric supply system 1. These pre-stored data can relate to a total amount of charge, so that the introduction of charge after an activation is continued until the amount of charge introduced into the electrophoretic TDS 40 corresponds to the predetermined value. The stored values can, furthermore, also serve for a temporally controlled charge introduction. For example, in the storage devices values can be stored which determine a first amount of charge for a first charging interval, a time period up to the next charging interval, and an amount of charge for a second charging interval. By sequentially linking several charging intervals and intermittent time periods any kind of temporal sequence for the charge introduction into an electrophoretic transdermal application system 40 can be pre-set.

Through a detection of the state of the electric connection from the intermediate storage to electrophoretic transdermal application system 40, e.g. by means of the output potential of the Schmitt trigger, the software-controlled device can determine the number of discharging cycles and, by summing up the same, the amount of charge transmitted to the electrophoretic TDS 40. Alternatively, the determination of the emitted amount of charge can also be performed, as described above, by a simple measurement of time using the known charging current for charging the capacitor.

In a preferred embodiment of the control unit 50, the amount of charge transmitted to an electrophoretic transdermal application system 40 is separately calculated by each of the two sub-units of the software-controlled device, with each of the two sub-units being adapted for interrupting a further charge supply to the electrophoretic TDS 40 when the amount of charge specified to be transmitted in total or for a partial time period has been reached.

The diagram 100 of FIG. 6 shows the results of an in-vitro mouse skin permeation test, in which an above-described electrophoretic transdermal application system is applied to a mouse skin, and the amount of active agent Fentanyl transported out of the system 40 through the mouse skin was measured. The curves 101 to 105 show the accumulated amount of permeated active agent, i.e. the amount that has penetrated the mouse skin up to the respective point of time.

The individual curves 101 to 105 differ from each other in the underlying test conditions. Each one of the curves represents the mean value of three tests with identical test conditions. The curve 105 represents the permeation result of a reference measurement where the charge was introduced into the electrophoretic transdermal application system 40 for 60 s at an electrode voltage of 2.5 V. The amount of charge introduced in this case into the electrophoretic transdermal application system 40 was 750 mC after having applied the 1st voltage (17 h), and 600 mC after having applied the 2nd voltage (41 h).

The test conditions for the skin permeation tests represented by the curves 101 to 104 differ from each other either in the capacity of the intermediate storage 30 or in the charging current with which energy was transferred from the primary energy storage 10 into the intermediate storage.

For the skin permeation test for determining the curve 101 and the curve 103 an intermediate storage with a capacity of 34.7 μF was used. In the skin permeation tests for determining the curves 102 and 104 the capacity of the intermediate storage was 970 μF. The charging current for the tests for determining the curves 103 and 104 was 1.8 times the charging current used for performing the tests for determining the curves 101 and 102.

The amount of charge transferred from the intermediate storage 30 to the electrophoretic transdermal application system 40 per discharge cycle was 34.7 μC for the tests as to curve 101, 970 μC as to curve 102, 34.7 μC as to curve 103 and 969 μC as to curve 104. The frequency of the discharge cycles was 52.6 Hz for curve 101, 1.85 Hz for curve 102, 29.7 Hz for curve 103 and 1.04 Hz for curve 104. From this, there results an amount of charge of 1.825 mC introduced per second into the electrophoretic transdermal application system 40 for the test regarding curve 101, 1.794 mC for curve 102, 1.031 mC for curve 103 and 1.008 mC for curve 104. In all four tests an amount of charge of altogether 600 mC was introduced into the electrophoretic transdermal application system 40. The period of time necessary for the introduction of this amount of charge was 5 minutes and 29 seconds for the test according to curve 101, 5 minutes and 34 seconds for the test according to curve 102, 9 minutes and 42 seconds for the one according to curve 103, and 9 minutes and 55 seconds for the one according to curve 104. In all tests, the charge transfer was activated 17 hours after the start of the test. The start of the charge application is marked by a vertical dashed line in FIG. 6.

The results of the permeation tests show comparable permeation profiles, with the initially steeper rise of the curve 105 belonging to the reference measurement can be put down to the introduced amount of charge being higher by 25%. The permeation profiles show a direct correlation with the charge transfer to the electrophoretic transdermal application system 40, and the time delay between the charge application and the rise of the permeation profile is to be put down to the time necessary for penetrating the mouse skin.

In FIG. 7 the basic steps of a method 200 for transferring a charge from a primary energy storage into an electrophoretic transdermal application system as described above are shown. The method starts in step 201 with the activation of the system by a user, e.g. a doctor, a medical assistant or by the patient himself/herself. Following the activation of the system, in the subsequent step 202 a charge is transferred from the primary energy storage 10 into the intermediate storage 30, with the current intensity with which the charge is transferred being smaller than or equal to the discharge current admissible for the used primary energy storage. In the next step 203 the state of charge of the intermediate storage is checked. If a first threshold value has been reached or exceeded, the process is continued with step 204, otherwise with step 202. In step 204 the intermediate storage 30 is electrically connected with the electrodes of the electrophoretic transdermal application systems 40, so that charge from the intermediate storage 30 can enter the reaction medium of the electrophoretic transdermal application systems 40. After having established the electric connection, the method is continued in step 205, in which it is checked whether the state of charge of the intermediate storage 30 has dropped to a second threshold value or has come below the same. If this is the case, the method is continued in step 206 with the interruption of the electric connection. In subsequent step 207 it is checked whether the amount of charge altogether transferred to the electrophoretic TDS 40 is equal to or larger than a predetermined amount of charge. If this is not the case, the method returns to step 202, otherwise it ends in step 208.

Instead of returning from step 207 to step 202 the method can also be continued by step 203 after step 207, if the current withdrawal from the primary energy storage 10 is maintained during the existence of the electric connection from the intermediate storage to the electrophoretic transdermal application system 40. Further embodiments of the method show steps not shown in FIG. 7, e.g. method steps, for checking the time that has passed since a prior charge application, so that a sequence of temporally spaced charge applications is possible. In these embodiments, the amount of charge introduced into the electrophoretic TDS 40 per application of charge can be variable.

1. A system for controlling an electrolytic reaction in a reaction medium, the system
being adapted to connect a primary energy storage, and
comprising an intermediate storage and
a control unit,
the control unit being adapted for
transferring electric energy from a connected primary energy storage into the intermediate storage,
monitoring the state of charge of the intermediate storage,
establishing an electric connection between the reaction medium and the intermediate storage when the state of charge of the intermediate storage is larger than or equal to a first threshold value, and
interrupting an electric connection between the reaction medium and the intermediate storage when the state of charge of the intermediate storage is smaller than or equal to a second threshold value, so as to interrupt the current flow from the system to the reaction medium.

2. A system for controlling an electrolytic reaction in a reaction medium, the system comprising
a primary energy storage,
an intermediate storage and
a control unit,
the control unit being adapted for
transferring electric energy from the primary energy storage into the intermediate storage,
monitoring the state of charge of the intermediate storage,
establishing an electric connection between the reaction medium and the intermediate storage when the state of charge of the intermediate storage is larger than or equal to a first threshold value, and
interrupting an electric connection between the reaction medium and the intermediate storage when the state of charge of the intermediate storage is smaller than or equal to a second threshold value, so as to interrupt the current flow from the system to the reaction medium.

3. The system according to claim 1, wherein a galvanic cell or an array of galvanic cells forms the primary energy storage.

4. The system according to claim 3, wherein one or more lithium manganese dioxide cells form the primary energy storage.

5. The system according to claim 3, which further comprises an energy transfer device which is adapted for transferring electric energy from the primary energy storage into the intermediate storage, with a charging current not exceeding a predetermined value.

6. The system according to claim 5, wherein the energy transfer device is adapted for transferring electric energy from the primary energy storage into the intermediate storage with a constant charging current.

7. The system according to claim 1, wherein the control unit is further adapted for determining the state of charge of the intermediate storage based on a voltage present at the intermediate storage.

8. The system according to claim 7, wherein the first and the second threshold value for the state of charge of the intermediate storage depend on the operating voltage of the primary energy storage.

9. The system according to claim 8, wherein the control unit is adapted for interrupting an electric connection between the reaction medium and the intermediate storage, when the second threshold value falls below a predetermined threshold value.

10. The system according to claim 9, wherein the control unit is adapted for determining a falling below of the predetermined threshold value on the basis of the operating voltage of the primary energy storage.

11. The system according to claim 6, which further comprises a reference voltage generator (V_{ref}) for generating a constant reference voltage independent of an operating voltage of the primary energy storage.

12. The system according to claim 11, wherein the energy transfer device is adapted for using the reference voltage in order to control the charging current.

13. The system according to claim 11, wherein the control unit is adapted for determining the operating voltage of the primary energy storage by use of the reference voltage.

14. The system according to claim 1, wherein a capacitor forms the intermediate storage.

15. The system according to claim 1, wherein the control unit is adapted for determining the amount of charge transferred from the intermediate storage into the reaction medium during an electric connection between the reaction medium and the intermediate storage.

16. The system according to claim 15, which further comprises a storage device for storing data that relate to the amount of charge introduced into the reaction medium during the electric connection(s) between the reaction medium and the intermediate storage.

17. The system according to claim 1, which further comprises a user interface adapted for activating the system through a user.

18. The system according to claim 17, wherein the user interface is further adapted for displaying information to the user.

19. An electrophoretic transdermal application system comprising:
a system for controlling an electrolytic reaction according to claim 1,
a first electrode,
a second electrode, and
a matrix located between the first electrode and the second electrode, which comprises at least one active agent, the ratio of which from its neutral to its ionic form changes with the pH value of the matrix, wherein the second electrode is adapted to be permeable for the at least one active agent in its neutral form, and for controlling an electrolytic reaction the system is electrically connected to the first electrode and the second electrode in such a way that the electric connection of the system to at least one of the electrodes is closed and interrupted by the control unit of the system in dependence on the state of charge of the intermediate storage.

20. A method for transferring electric charges from a primary energy storage into a transdermal application system, which comprises the following steps:
(a) transferring electric charges from a primary energy storage into an intermediate storage at a transfer rate not exceeding a predetermined value,
(b) comparing the state of charge of the intermediate storage with a first predetermined state of charge,
(c) establishing an electric connection between the intermediate storage and the transdermal application system when the state of charge corresponds to or exceeds the first predetermined state of charge,
(d) comparing the state of charge of the intermediate storage to a second predetermined state of charge,
(e) disconnecting the electric connection between the intermediate storage and the transdermal application system when the state of charge corresponds to or falls below the second predetermined state of charge.

21. The method according to claim 20, wherein the steps (a) to (e) are performed repeatedly until a predetermined amount of charge has been transferred.

22. A method comprising using a system according to claim 1 for controlling an electrolytic reaction in a transdermal application system.
